(19) United States
(12) Patent Application Publication
(10) Pub. No.: US 2008/0026084 A1
(43) Pub. Date: Jan. 31, 2008

(54) Method and composition for management of weight and blood sugar

(75) Inventor: Ven Subbiah, Greenville, NC (US)

Correspondence Address: WARD AND SMITH, P.A., 1001 COLLEGE COURT, P.O. BOX 867, NEW BERN, NC, 28563-0867, US

(73) Assignee: PhytoMyco Research Corporation, Greenville, NC (US)

(21) Appl. No.: 11/880,701

(22) Filed: Jul. 24, 2007

Related U.S. Application Data
(60) Provisional application No. 60/833,165, filed on Jul. 25, 2006.

(51) Int. Cl. A61K 36/42; A61P 3/04; A61P 3/10; C12Q 1/02; C12Q 1/32

(52) U.S. Cl. 424/725; 435/26; 435/29

(57) ABSTRACT

A method for the identification of a composition useful in the treatment of an overweight or obese person to reduce the person's body mass index is provided which comprises obtaining an extract of an ethnobotanical plant, and evaluating the activity of the extract in an assay. A composition is described for treating patient conditions made up of C. grandis standardized extract (AdipoCleave™) with cucurbitacins B and D as active ingredients, Cephalandrol, Cephalandrine A and B, and like compounds and cucurbitacins. The composition is effective to maintain normal blood sugar and normal levels of non-enzymatic protein glycosilation.

### CROSS REFERENCE TO RELATED APPLICATION

This application is related to and claims priority to U.S. Provisional Application Ser. No. 60/833,165 filed Jul. 25, 2006, entitled “Method of Management of Weight and Blood Sugar”. The disclosure of that provisional application is specifically incorporated by reference herein in its entirety.

### BACKGROUND OF THE INVENTION

1. Field of the Invention

This invention relates to dietary supplement compositions and methods of use of such compositions generally. More particularly, the invention relates to compositions and methods useful for reducing the rate of weight gain in an overweight and/or obese person; to compositions and methods for effecting weight loss in an overweight and/or obese person; to compositions and methods useful to reduce the body mass index of an overweight and/or obese person or animal, to compositions and methods useful for reduction and control of weight in the treatment of persons who are overweight or obese; and to methods of discovering compounds and compositions useful in the treatment of obesity and/or useful in to produce a reduction in body mass index of an overweight or obese person.

In an alternative aspect, the invention also relates to dietary supplement compositions and methods of use thereof generally, and in particular to compositions and methods useful for reducing the blood sugar level as well as increasing the insulin secretion in diabetic persons; to compositions and methods for affecting blood glucose level in an overweight and/or obese person; to compositions and methods useful to reduce the blood sugar content of an overweight and/or obese person or animal; to compositions and methods useful for reduction and control of blood sugar content in the treatment of persons who are overweight and diabetic; and to methods of discovering compounds and compositions useful in the treatment of diabetes and/or useful to produce a reduction in blood sugar content of an overweight or obese or diabetic person.

In a yet still more specific aspect, the invention relates to the field of dietary supplement compositions derived as extracts from ethnopharmacological plants, to methods useful to identify standardized extract in such extracts that are active in the treatment of obesity, diabetes and overweight conditions, and especially to compositions comprising of standardized extract (AdipoCleave™) of Coccinia grandis plants and the compounds, cucurbitacin B and/or cucurbitacin D, Cephalandrol, Cephalandrine A and B and their use in the treatment of overweight, diabetes and obese persons, such as by oral administration.

2. Discussion of Relevant Art

Body Mass Index (BMI) has been recognized by the U.S. Department of Health as a reference relationship between a person's height and weight. To determine a person's Body Mass Index in kg/m², the weight of the person, for example in pounds, is first multiplied by a conversion factor of 703 (with units of kilogram.inch².pound⁻¹.meter⁻²), and that result is then divided by the height of the person, in inches squared. Alternatively, when height is measured in meters (1 inch=0.0254 meters) and weight in kilograms (1 pound Avoirdupois (U.S.)=0.4536 kg), the conversion factor is unity, and the Body Mass Index is obtained directly in units of kg/m².

Body Mass Index (BMI) can be used to determine when extra weight above an average or normal weight range for a person of a given height can translate into and signal increased probability for additional health risks for that person. Such health risks are considered to be related to the presence of additional weight above a desired range for that person.

Body Mass Index does not directly measure percent of body fat, although higher BMI's are usually associated with an increase in body fat, and thus excess weight. A BMI of 24 kg/m² or less is a commonly accepted guideline used to define a person with a healthy weight and who is neither overweight or obese. A desired BMI range is from about 18 kg/m² to about 24 kg/m², wherein a person is considered to have a healthful weight for the person's height. A Body Mass Index reading under 20 kg/m², and especially under 18 kg/m², is frequently considered to signify that the person is underweight which can be unhealthy. A person with a BMI above 24 kg/m², such as from about 25 kg/m² to about 30 kg/m², is frequently considered to be overweight, and a person with a BMI above about 35 kg/m² is frequently considered to be obese In another aspect, an individual who has a BMI in the range of about 25 kg/m² to about 35 kg/m², and has a waist size of over 40 inches for a man and over 35 inches for a woman, is considered to be at especially high risk for health problems.

Health risks related to a person being overweight and health risks related to a person being obese represent rapidly growing threats to the health of populations in an increasing number of countries worldwide. Obesity is a disease that is prevalent in both developing and developed countries and that affects children and adults alike.

Information available from the American Heart Association related to statistical results of the Third National Health and Nutrition Examination Survey 1988-1994 suggests that among American children aged 6 to 11 years, the following percentages were overweight, using 95th percentile of body mass index (BMI) values: for non-Hispanic whites, 10.3 percent of boys and 9.2 percent of girls were overweight; for non-Hispanic blacks, 11.9 percent of boys and 16.4 percent of girls were overweight; and for Mexican Americans, 17.4 percent of boys and 14.3 percent of girls were overweight.

Among adolescents aged 12 to 17 years, the following percentages were overweight, using the 95th percentile of BMI values: for non-Hispanic whites, 11.1 percent of boys and 8.5 percent of girls were overweight; for non-Hispanic blacks, 10.7 percent of boys and 15.7 percent of girls were overweight; and for Mexican Americans, 14.6 percent of boys and 13.7 percent of girls were overweight.

Among adults aged 18 and older, the following people are overweight (defined as a body mass index (BMI) of 25 kg/m² or higher): for non-Hispanic whites, over 62 percent of men and over 43 percent of women are overweight; for non-Hispanic blacks, over 64 percent of men and over 64 percent for women are overweight; for Hispanics, over 64 percent of men and over 56 percent of women are overweight; for non-Hispanic Asian/Pacific Islanders, over 35 percent of men and over 25 percent of women are overweight.

Among Americans aged 18 years and older, the median percentages of obesity are (defined as a body mass index or BMI greater than 30 kg/m²): for whites, about 15 percent; for blacks, about 26 percent; for Hispanics, about 18 percent; for Asian/Pacific Islanders, about 4.8 percent; and for American Indians/Alaska Natives, about 30 percent.

Among Americans aged 20 to 74 years, an age-adjusted prevalence of being overweight (i.e., having a BMI of 25.0 kg/m² or higher) and of being obese (i.e., having a BMI of 30.0 kg/m² or higher) are: for non-Hispanic whites, about 61 percent of men and about 47 percent of women are overweight, and about 21 percent of men and about 23 percent of women are obese; for non-Hispanic blacks, about 58 percent of men and about 68 percent of women are overweight, and about 21 percent of men and about 38 percent of women are obese; for Mexican Americans, about 69 percent of both men and women are overweight, and about 25 percent of men and about 36 percent of women are obese.

Among American Indians aged 45 to 74 years, about 26 percent of men and about 31 percent of women are overweight (defined as a BMI of about 28 kg/m² to about 31 kg/m² for men and about 27 kg/m² to about 32 kg/m² for women), and about 35 percent of American Indian men and about 41 percent of women are obese (defined as a BMI of about 31 kg/m² or higher for men and a bout 32 kg/m² or higher for women).

The year-to-year rate of increase in the number of overweight children and adolescents is about 2 to 3 percent. Each year an estimated 300,000 U.S. adults die of causes related to the health risk of obesity. Over 100 million American adults (over 56 million men and over 52 million women) are overweight with a BMI of about 25 kg/m² and higher. Of these, over 44 million American adults (over 18 million men and over 25 million women) are obese, having a body mass index (BMI) of about 30 kg/m² or higher. (Obesity Clinical Guidelines: NIH Statement Jun. 3, 1998, press release).

A person can be overweight but not obese if that person's BMI is between about 25 to 30 kg/m², while a person who is obese with a BMI above 30 kg/m² is also overweight.

In one aspect, in order to lessen the risk for health problems in an overweight person and to improve that person's health, it is desirable to provide compositions and methods useful in the treatment of an overweight person which produces a reduction in the person's body mass index (BMI), preferably from a level above 25 kg/m² to a level below 25 kg/m², and more preferably to a BMI level between 25 kg/m² and 18 kg/m².

In another aspect, in order to lessen the risk for health problems in an obese person and to improve that person's health, it is desirable to provide compositions and methods useful in the treatment of an obese person which produce a reduction in the person's body mass index (BMI), preferably from a level above 30 kg/m² to a level below 30 kg/m², more preferably to a level below 25 kg/m², and most preferably to a BMI level between 25 kg/m² and 18 kg/m².

A person who has a BMI of between about 30 kg/m² to about 25 kg/m² is considered to be overweight, and a person who has a BMI of greater than about 30 kg/m² is considered to be clinically obese. Both genetic and environmental factors can contribute to a person becoming overweight or obese. The most common cause of weight gain that can produce elevations in a person's BMI that are synonymous to being overweight and/or obese is a high caloric food intake especially in the absence of exercise or physical activity. The resulting accumulation of surplus fat places overweight or obese individuals at increased risk of illness such as hypertension, lipid disorders, type 2 diabetes, cardiovascular diseases, high blood pressure, elevated levels of cholesterol, hyperlipidemia, coronary heart disease, stroke, gallbladder disease, osteoarthritis, joint pain, sexual and fertility problems, sleep apnea and respiratory problems, skin conditions, certain type of cancers, and a wide variety of other diseases and undesired physiological conditions, as well as overall mortality. An obese individual with a BMI above about 30 kg/m² is up to about fourteen times more likely to die at a significantly younger age than a lean person with a BMI below 25 kg/m². Obesity creates a high-risk medical burden on society and its treatment would be highly desirable. Eliminating or reducing obesity would also decrease medical costs for treating many co-morbid conditions.

The biochemical mechanisms related to adipogenesis, to becoming overweight and to becoming obese are complex and comprise a number of interactions between ligands and their receptors, such as leptin, NPY monoamines (dopamine, serotonin, and norepinephrine), and CART (i.e., cocaine amphetamine regulated transcript), and others.

The obesity phenotype is probably connected to over 250 genes, markers and chromosomal regions in the human genome. Commonly used markers for adipocyte differentiation include aP2 (fatty acid binding protein), GPDH (glycerol-3-phosphate dehydrogenase), and adipsin. Regulatory factors that influence adipogenesis by influencing transcription include the CAAT enhancer-binding proteins a, b, d (C/EBP a,-b,-d), peroxisome proliferator-activated receptors (e.g., PPARgamma), sterol response element binding proteins (SREBP), and preadipocyte factor 1 (pref-1). The progression of adipocyte differentiation can be characterized by a number of genetic changes involving expression of early, intermediate, and late genes.

Adipogenesis is a multistep organogenenic process that begins in the prenatal period, but unlike osteogenesis and myogenesis, the adipogenesis process never ends. In this process, mesenchymal cells can proliferate in clonal expansion, and at some point, some of these cells can differentiate into preadipocytes or cells committed to fill with lipid (i.e., fat) and then become adipocytes. When preadipocytes undergo a differentiation step and begin to fill with lipid, lipid first accumulates within the cell in small droplets (multilocular cells) and eventually the droplets fuse into one large droplet (unilocular cells). The adipocyte can continue to enlarge by accumulating additional lipid. A typical mesenchymal cell is 10 to 20 μm in diameter, but adipocytes can easily reach 100 μm (and in some cases 200 μm) in diameter. The volume of the cell can increase as much as a thousand fold largely because of lipid accumulation.

Obesity is the result of numerous, interacting behavioral, physiological, and biochemical factors. One increasingly important factor is the generation of additional fat cells, or adipocytes, in response to excess feeding or intake of food, especially food high in fat or comprising fat or fat pre-metabolites, and/or large increases in body fat composition. The generation of new adipocytes is controlled by several adipocyte-specific transcription factors that regulate preadipocyte proliferation and adipogenesis. Generally these adipocyte-specific factors are expressed following the induction of adipogenesis.

Reusch et al. in Mol Cell Biol. February 2000; 20 (3): 1008-1020 noted that transcription factor(s) involved in initiating adipocyte differentiation had not been identified, but described how the transcription factor, CREB, was constitutively expressed in preadipocytes and throughout the differentiation process. They noted that CREB was stimulated by conventional differentiation-inducing agents such as insulin, dexamethasone, and dibutyryl cAMP. Stably transfected 3T3-L1 preadipocytes (L1 cells) were generated in which they could induce the expression of either a constitutively active CREB (VP16-CREB) or a dominant-negative CREB (KCREB). Inducible expression of VP16-CREB alone was sufficient to initiate adipogenesis as determined by triacylglycerol storage, cell morphology, and the expression of two adipocyte marker genes, peroxisome proliferator activated receptor gamma 2, and fatty acid binding protein. KCREB alone blocked adipogenesis in cells treated with conventional differentiation-inducing agents. These data suggested that activation of CREB was necessary and sufficient to induce adipogenesis. CREB was shown to bind to putative CRE sequences in the promoters of several adipocyte-specific genes. These data also suggested CREB as a primary regulator of adipogenesis.

Insulin can play a role in controlling adipogenesis. Several factors exert positive and negative influences on the process of adipogenesis. When insulin binds to its receptor it causes a signal cascade that ultimately leads to the translocatation of GLUT-4 receptors to cell surface membranes to allow glucose to be taken up by the cells.

Understanding weight regulation involves, in part, unraveling the roles played by insulin and leptin in regulating appetite control and energy expenditure in the brain. Both of these blood-borne signals provide information to the brain about fat storage. Homeostasis and glucose metabolism is ultimately regulated in the hypothalamus, specifically in the arcuate nucleus (ARC) region. Two subsets of neurons in the ARC express receptors for insulin and leptin and are believed to effect metabolism through an anabolic/catabolic regulation pathway. The proposed catabolic circuit involves the proopiomelanocortin (POMC) expressing neurons. These are activated by insulin and leptin causing the release of the neuropeptide -MSH, believed to increase energy expenditure and reduce food intake. Neuropeptide Y (NPY) and agouti related protein (AgRP) expressing neurons complete the anabolic circuit becoming activated by low levels of insulin and leptin, stimulating downstream neurons to increase food intake and energy storage (i.e., increase insulin levels). Insulin and leptin satisfy criteria required for adiposity signals: secretion into plasma in proportion to body fat stores, transport into the brain from the bloodstream, expression of signal-transducing molecules in brain areas that control energy homeostasis, and the ability to reduce food intake upon central administration.

One potential cause of obesity is a resistance or desensitization of receptors to insulin and leptin in the blood, which results in an increase of food intake and body adiposity as well as glucose intolerance, hyperleptinemia, and reproductive abnormalities. Several factors can play a role in determining cell sensitivity to insulin.

Thiazolidinediones can increase insulin sensitivity, while several substances including tumor necrosis factor (TNF-alpha), growth hormone, plasminogen-activator inhibitor-1, angiotensin 2, free fatty acids, and the hormone resistin can decrease insulin sensitivity. Insulin can up-regulate resistin gene expression, possibly serving as a negative feedback loop, while isoproterenol, the cytokine TNF-alpha and activation of alpha-adrenoceptors suppress resistin expression and secretion. TNF-alpha can act as a regulator of adipocytes, and may be involved in the development of Type II diabetes as well as obesity.

Available treatments for obesity can produce undesirable and serious side effects or such treatments may lack efficacy. Obesity and overweight conditions may become partially reversed or prevented by employing diet or nutrition and behavior modification programs or by administration of pharmaceutical therapeutic (drug) compositions. Widely administered drugs include orlistat, which reduces the amount of dietary fat that is absorbed from the intestine; sibutramine, which suppresses appetite by inhibiting the re-uptake of norepinephrine and serotonin; fenfluramine and d-fenfluramine, which suppress appetite by both releasing serotonin and then inhibiting its re-uptake; and phentermine, which suppresses the appetite by stimulating the release of norepinephrine.

Drug therapies for weight reduction usually achieve only a 5% to 10% decrease in body weight (National Task Force on the Prevention and Treatment of Obesity: Long-term pharmacotherapy in the Management of Obesity, JAMA 276:1907-15, 1996). Many drugs produce mild to serious side effects in overweight and obese patients. Common side effects include dizziness, headaches, rapid pulse, palpitations, sleeplessness, hypertension, diarrhea, and intestinal cramping. The combination of fenfluramine and phentermine, which produced a 15% to 20% reduction in body weight (F. Brenot et al., Appetite Suppressant Drugs and the Risk of Primary Pulmonary Hypertension, N. Engl. J. Med., 335:609-16, 1996), also provided an increased the risk of heart valve damage and a number of confirmed patient deaths related to “Fen-Phen”. Medications used for weight loss can fall into two groups: those that reduce the absorption of nutrients into the body and those that reduce appetite and thereby decrease food intake.

Adipose tissue produces leptin that reaches homeostatic hypothalamic centers in the brain and provides information on the state of the energy balance. Adipocytes play a critical role in the storage of energy as lipid and in the overall regulation of the body's metabolism. That adipocytes are endocrine cells that have a profound effect on human physiology has been postulated for over 40 years. Recent studies indicate that obesity affects about 30% of the population and that obesity has been identified as a risk factor for metabolic diseases such as type 2 diabetes, hypertension, and hyperlimidia. A better understanding of human adipocyte biology is critical to the development of pharmacological agents to treat these devastating diseases.

Orlistat (branded under the trademark Xenical) was approved by the FDA for reducing nutrient absorption. Agonasts are known to induce lipolysis, and thereby have the potential to offer weight-lowering properties. Beta-agonists, such as isoproterenol and turbataline, have been shown to increase lipolysis, leading to an increase in energy expenditure and a decrease in fat stores. Beta-agonists can also modulate glucagon and insulin secretion, liver metabolism and glucose uptake in muscle via beta-adrenergic receptors (beta2adrenergic receptor, beta3adrenergic receptor). In addition, polymorphisms of the beta3adrenergic receptor may be involved in insulin resistance and hypoglycemeia. However, use of beta-agonist can also lead to hyperglycemia, or an increase in blood sugar levels as well as insulin desensitization.

A number of weight loss drugs have been marketed, but most have undesired side effects for the patient. For example, a combination of amphetamine and dextroamphetamine, branded under the trademark Adderall, is a sympathomimetic amine appetite suppressant with high abuse potential. Benzphetamine, branded under the trademark Didrex or Benzfetamine, is a sympatho—mimetic amine appetite suppressant also with high abuse potential. Bromocriptine, branded under the trademark Ergoset or Parlodel, stimulates dopamine type-2 receptors and antagonizes type-1 receptors in brain, but it is not approved for treatment of obesity although it is used off label. Dexfenfluramine, branded under the trademark Redux, acts as an appetite suppressant via serotonin release and serotonin reuptake block, but it was voluntarily withdrawn from the market because of evidence of heart valve damage. Dextroamphetamine, branded under the trademark Dexedrine, is a sympathomimetic amine appetite suppressant used off-label for obesity although it is highly abused. Diethylpropion, branded under the trademark Amfepramone and Tenuate, is a sympatho-mimetic amine appetite suppressant with a possible link to primary pulmonary hypertension. Fluoxetine, branded under the trademark Prozac, is a selective serotonin reuptake inhibitor (SSRI) that is used off-label. Mazindol, branded under the trademark Mazanor and Sanorex, is a sympathomimetic amine appetite suppressant subject to high abuse potential. Methamphetamine, branded under the trademark Desoxyn or Methampex, is a sympathomimetic amine appetite suppressant subject to high abuse potential. Orlistat, branded under the trademark Xenical, is not a CNS-active drug but can decrease the amount of fat absorbed from the diet by about 30%. However, the drug may have a link to breast cancer. Other sympathomimetic amine appetite suppressants include Phendimetrazine, Phentermine which was approved as a resin complex, and Phenylpropanolamine which is available over the counter. Additionally, Sibutramine, branded under the trademark Meridia inhibits reuptake of dopamine, norepinephrine, and serotonin in the brain.

In addition to the above types of drug therapeutic agents, a number of herbal weight reduction formulas have also been suggested as alternatives to both prescription and over-the-counter weight loss compounds. Formulations containing herbal components can have fewer side effects than prescription and over-the-counter medications, but some herbal formulas can still be abused. For example, improper administration of herbal weight loss formulas based primarily on ma huang (ephedra) and high caffeine-containing herbs, such as guanrana and kola nut, may result in diminished energy and a depleted body.

U.S. Pat. No. 6,541,046 describes an herbal composition for hindering weight gain comprising: rhubarb, turmeric, astragalus root, red sage root, and ginger root.

U.S. Pat. No. 6,322,823 describes an aromatherapy composition directed to combat symptoms of premenstrual syndrome (PMS) comprising externally applied highly concentrated essential oils extracted from plant cells. The composition can comprise a mixture of essential oil of geranium, essential oil of clary sage, and essential oil of orange, and can be applied directly on the skin or in a carrier.

Given the prevalence and serious problems associated with obesity, and the significant drawbacks associated with many weight loss compounds, a need exists for a means to discover compounds and compositions that are safe and effective in the treatment of persons that are overweight or obese to reduce weight gain, cause weight loss, and reduce body mass index to acceptably healthy levels in the range of 25 to 18 kg/m².

In the body, sugar(s) have specific functions. They are the primary substrate for immediate energy. In addition, sugars are a necessary component of, for example, collagen, the body's most abundant connective tissue. Collagen provides the foundation for proper form and function of skin, blood vessels, and numerous other organs.

Blood sugar refers to plasma glucose and is typically expressed as milligrams of glucose per deciliter (mg/dl) of blood. Glucose is a primary cellular energy source and one of the most well controlled substances in the body. When held in its normal healthy range, proper form and function of, for example, collagen is maintained. Plasma glucose rises and falls with eating and physical exertion, a change in metabolic demand, and emotional stress. Fasting plasma glucose concentration and tolerance to a dose of glucose are used to establish normal glucose utilization and disposal. Fasting plasma glucose is usually measured at least 8 but not more than 16 hours postprandial. The healthy normal range for fasting plasma glucose is 60-109 mg/dl. Fasting plasma glucose levels of more than 126 mg/dl can be indicative of a metabolic or endocrinological disease or disorder, stress (emotional, oxidative, and metabolic), or impaired utilization of glucose, for example diabetes.

Plasma glucose can be measured by any means known in the art (finger-stick and capillary blood placed on test strip; withdrawal of venous blood placed on a slide analyzed by computer), for example, The Vitros Test Methodology [Ortho-Clinical Diagnostics, 100 Indigo Creek Drive, Rochester, N.Y. 14626]. After an overnight fast (at least 8 hours), a single sample of blood is drawn and analysed using standardized equipment such as the vitros analyzer. Vitros GLU slides quantitatively measure glucose (GLU) concentration in serum, plasma, and other body fluids. The Vitros GLU Slide is a dry, multilayered, analytical element coated on a polyester support. A 10 .mu.L drop of a sample of blood is deposited on the slide where the spreading layer promotes the uniform distribution of the sample and permits an even penetration of solute molecules into the underlying reagent layer. The oxidation of sample glucose is catalyzed by glucose oxidase to form hydrogen peroxide and gluconate. This reaction is followed by an oxidative coupling catalyzed by peroxidase in the presence of dye precursors to produce a dye. The intensity of the dye is measured by reflected light.

### BRIEF SUMMARY OF THE INVENTION

In one aspect, the invention relates to a method of treatment of diabetes and obesity in a patient with a BMI of 30 kg/m² or greater. The method involves administering to the patient C. grandis standardized extract (Adipo Cleave) with curcurcibitacins B and D, and Cephalandrol, Cephalandrin A and B as active ingredients.

In an alternative aspect, there is provided a method of treatment of a high blood sugar content condition, similar to the conditions of diabetes or being overweight, in which the aforementioned components are administered such that the BMI of the patient is reduced from a range of about 30 kg/m² to about 25 kg/m² -24 kg/m². Yet still further, the invention involves a method of treatment of diabetes, including administering the aforementioned component for reducing the BMI of the patient to a value of 30 kg/m² or less.

In an alternative aspect, there is provided a healthy blood sugar maintenance composition, which is made of an effective amount of C. grandis standardized extract (AdipoCleave) with curcurcibitacins B and D as active ingredients, and Cephalandrol, Cephalandrin A and B and related analogs.

In a yet still further and alternative aspect, there is provided a process for identification of a composition or compound useful in the treatment of a diabetic or overweight or obese person. The process for identifying the composition includes an assay made up by obtaining an extract of a ethnobotanical plant and evaluating the activity of the extract in an assay selected from the group made up of a lipolysis assay, an assay that measures the amount of glycerol introduced by cell into a suspension medium of the cell, an adipocyte differentiation assay, an assay that measures the level of the enzyme glycerol-3-phosphate dehydrogenase, an assay that measures the inhibition of differentiation of preadiphocytes to adiphocytes, an assay that measures the accumulation of lipid in an adiphocyte, an assay that measures the differentiation of adiphocytes into pre-adiphocytes, in combinations thereof.

In more specific aspects, the present invention provides compositions (pharmaceutical, nutraceutical, or both), including compounds and formulations, and methods useful in the treatment of a wide variety of physiological and mental disorders and abnormal conditions, including those disorders and conditions related to being over a normal healthy weight for body height (over weight), body mass higher than normal and healthy body mass for a given height (high body mass), blood sugar levels higher than observed in a normal and healthy individual (high blood sugar), and cholesterol levels above the range observed in normal and healthy individuals (hypercholesterolemia or high cholesterol) in humans and animals. The compositions (extracts, mixtures, isolated compounds, and formulations of these) and methods of the present invention are useful in treating undesired or unhealthy conditions and/or disorders such as over weight and other metabolic diseases, such as high blood sugar, diabetes, hypertension, pulmonary hypertension, hyperlipidemia, hypercholesterolemia, hyperlipoproteinemia, and the like.

Little gourd [Coccinia grandis (Linn) Voight], Cucurbitaceae (Synonym Coccinia indica L.) and standardized extract, AdipoCleave™, cucurbitacin B-like compounds and cucurbitacin D and cucurbitacin D-like compounds Cephalandrol, Cephalandrine A and B-like compounds can be formulated and used in combination with a pharmaceutically and nutraceutically acceptable carrier, such as a carrier comprising a pharmaceutically acceptable excipient and/or diluent, to provide a pharmaceutical and/or nutraceutical composition suitable for use in treatment of a one or more diseases related to conditions or overweight and obesity. In one embodiment, C. grandis standardized extract with cucurbitacins as active ingredients-like and cucurbitacins-like compounds can be used in the treatment of diabetes and obesity to achieve a reduction in body mass index in a patient, diabetes, hypercholesterolemia to achieve a lowering of cholesterol levels in the blood, and coronary heart disease.

Accordingly, the present invention relates to a method of treating abnormal conditions and disorders such as high blood sugar level, over body weight disorders including obesity and related conditions. In one embodiment, a method of the invention comprises administering to a subject an active compound of the present invention (i.e., a C. grandis standardized extract with cucurbitacins as active ingredients, cucurbitacins-like compound) in an amount sufficient to treat the disorders such as to achieve a reduction in body mass index, a reduction in cholesterol level, a reduction in low density lipid levels, and the like. The compounds and compositions of the present invention may be provided to the subject in a pharmaceutical or nutraceutical formulation, examples of which formulations are also described herein as embodiments of the invention. Another embodiment of the invention involves a method of using a C. grandis standardized extract with cucurbitacins as active ingredients, cucurbitacins-like compounds to treat or manage diabetes and/or to treat or manage coronary heart disease, particularly to treat or manage those disorders and conditions associated with adipose tissue abnormalities. Adipose tissue abnormalities.comprise risk factors directly related to metabolic abnormalities and diseases such as type 2 diabetes, hypertension, and hyperlipidemia.

There is a continuing demand for dietary supplements that are useful and effective in treatments to reduce blood sugar level, body weight and body mass index from overweight and obese levels to less overweight and ultimately to normal levels. Compositions of this invention have been assessed for utility in treatment of diabetes, overweight and obese conditions using cell-based assays, and animal models that are useful for detecting weight-lowering substances.

### BRIEF DESCRIPTION OF THE SEVERAL VIEWS OF THE DRAWINGS

FIG. 1 shows the effect, after 12 days in terms of measured GPDH levels, of C. grandis standardized extract (AdipoCleave) with cucurbitacins as active ingredients on adipogenesis in 3T3-L1 cells that were differentiated into adipocytes. Results are as percent inhibition compared to a positive control average shown as 0% inhibition.

FIG. 2 shows the effect, as a percentage of the untreated control average, of C. grandis standardized extract with cucurbitacins as active ingredients on the inhibition of adipogenesis via Oil Red O assay in 12-day old differentiated adipocyte treated with 40 ug/ml of cucurbitacins and AdipoCleave for 3 hours.

FIG. 3 shows the effect, after 12 days in terms of measured GPDH levels, of C. grandis standardized extract with cucurbitacins as active ingredients on adipogenesis in 3T3-L1 cells that were differentiated into adipocytes. Results are based on Microscopic observations.

FIG. 4 shows the effect, as a percentage of the untreated control average, of C. grandis standardized extract with cucurbitacins as active ingredients on lipolysis via glycerol release in 12-day old differentiated adipocytes treated with 40 ug/ml of cucurbitacins for 3 hours.

FIG. 5 is a the effect of AdipoCleave, the standardized extract of C. grandis on the leptin secretion using commercially available ELISA assay that can be used to detect compounds that induce the release of leptin in this invention.

FIG. 6 is a bar graph showing the blocking of adipocyte differentiation, as a percent of positive control, by representative compounds designated as “marker compounds” after 10-fold dilution, wherein Marker compound is cucurbitacins the marker compound from C. grandis standardized extract (AdipoCleave).

FIG. 7 is a graphical representation of the amount of lypolysis observed in the presence of representative compounds designated as “marker compounds” after 10-fold dilution, wherein Marker compound is cucurbitacins the marker compound from C. grandis standardized extract (AdipoCleave).

FIG. 8 is a table which describes the effectiveness of C. grandis standardized extract (AdipoCleave) with cucurbitacins as active ingredients as appetite suppressant in rat model.

FIG. 9 is a table which describes the effectiveness of C. grandis standardized extract (AdipoCleave) with cucurbitacins as active ingredients as blood sugar lowering agent as well as increasing the insulin content in rat model.

FIG. 10 is a table which describes the non-toxic of C. grandis standardized extract (AdipoCleave) with cucurbitacins as active ingredients when the glutathione content was measured in liver and kidney comparing with positive control, Glibenclamide.

FIG. 11 is a table which describes the non-toxic of C. grandis standardized extract (AdipoCleave) with cucurbitacins as active ingredients when the hydroperoxides and TBARS (Thiobarbituric acid reactive substances) content was measured in liver and kidney comparing with positive control, Glibenclamide.

### DETAILED DESCRIPTION OF THE INVENTION

Specific agonists of CART (cocaine amphetamine regulated transcript) peptide receptors can be useful in treating obesity.

3T3-L1 cells (L1 cells), derived from mouse embryos, are preadipocytes and can be induced to morphologically change into adipocytes (fat cells), which are detectable microscopically by the presence of oil droplets in the cytoplasm. Cell-based assays which employ the 3T3-L1 cell line can provide an in vitro model useful to study adipocyte differentiation and obesity. Cell-based assays which employ the 3T3-L1 cell line can also provide an in vitro model useful to detect substances and compounds that have an effect on obesity or weight loss.

Compounds that block differentiation of preadipocytes to adipocytes (fat cells) can be useful in that they can prevent the development of cells whose function is the accumulation and/or synthesis of stored triacylglyceride or fat. Because an adipocyte secretes a number of inflammatory cytokines, blocking the differentiation process can also result in less need for inflammatory mediators such as TNF-α, whose presence is correlated with insulin resistance.

Myostatin, an adipocyte blocking agent, is an inhibitor of skeletal muscle growth and is secreted from muscle tissue. Like other members of the TGF-alpha super family, myostatin inhibits differentiation of preadipocytes in 3T3-L1 cells. A decrease in GPDH (glyceraldehyde phosphate dehydrogenase) activity as well as morphological evidence (e.g., oil red O staining) shows that myostatin inhibits adipogenesis. On the molecular level, myostatin did not alter C/EBP alpha expression, but did cause a decrease in the expression of the downstream genes C/EBPalpha and PPAR gamma (Kim et al. 2001). Consequently, since C/EBPalpha activates the promoter for leptin, while PPAR gamma antagonizes activation of the promoter (He et al 1995), myostatin had no effect on leptin levels.

Several methods have been developed to detect the inhibition of adipocyte differentiation. Lipophilic dyes can be used to stain adipocytes such as Oil red O and Red Nile. Branched DNA (bDNA) analysis can be used to measure the mRNA levels in cells for adipocyte specific marker genes, such as AP2, a fatty acid binding protein specifically expressed in adipocytes. Its expression is controlled by the nuclear transcription factor peroxisome proliferator-activated receptor gamma (herein referred to as PPAR gamma). PPAR gamma is predominately expressed in adipose tissue and along with the transcription factors C/EBP and CCAAT/enhancer binding protein family) play a role early in adipose differentiation by regulating the promoters for several adipogenic genes.

Enzymes that are upregulated in fat cells can be utilized as markers for adipocytes, such as glyceraldehyde phosphate dehydrogenase (GPDH).

Triglyceride synthesis and accumulation is one of the events that signals late stage adipocyte differentiation and therefore cell maturity. It is also at this stage in differentiation that adipocytes acquire insulin sensitivity. Triglyceride synthesis and accumulation in adipocytes relate the role played by adipocytes as endocrine cells and stores of energy. Compounds that induce lipolysis may be used to reduce the storage of fat in the body. Additionally, such compounds may break down existing lipids/fat cells; or at least lower the fat content of cells. The breakdown of triglyceride in adipocytes leads to secretion of free fatty acid and glycerol into the medium. Understanding what causes the breakdown of triglycerides in the cell could have a profound impact on the development of antiobesity drugs.

Pharmaceutically acceptable vehicle” means a carrier, diluent, adjuvant, or excipient, or a combination thereof, with which a component is administered to a patient. A pharmaceutically acceptable vehicle may include, but is not limited to, polyethylene glycol; wax; lactose; glucose; sucrose; magnesium stearate; silicic derivatives; calcium sulfate; dicalcium phosphate; starch; cellulose derivatives; gelatin; natural and synthetic gums such as, but not limited to, sodium alginate, polyethylene glycol and wax; suitable oil; saline; sugar solution such as, but not limited to, aqueous dextrose or aqueous glucose; DMSO; glycols such as, but not limited to, polyethylene or polypropylene glycol; lubricants such as, but not limited to, sodium oleate, sodium acetate, sodium stearate, sodium chloride, sodium benzoate, talc, and magnesium stearate; disintegrating agents, including calcium carbonate, sodium bicarbonate, agar, starch, and xanthan gum; and absorptive carriers such as, but not limited to, bentonite and klonin.

“Therapeutically effective amount” means the amount of a component that is sufficient to at least partially effect a treatment of a condition when administered to a patient. The therapeutically effective amount will vary depending on the condition, the route of administration of the component, and the age, weight, etc. of the patient being treated.

By systemic administration is meant oral, while it is possible for an active ingredient to be administered alone as the raw chemical, it is preferable to present it as a pharmaceutical formulation. The active ingredient may comprise, for oral administration, from 0.01 to 5.0 wt % of the formulation.

For oral administration of one active ingredients in 5% dextrose in water or normal saline, or a similar formulation with suitable excipients, is most effective. Typically, the parenteral dose will be about 0.01 to about 50 mg/kg; preferably between 0.1 and 20 mg/kg, in a manner to maintain the concentration of drug in the plasma at a concentration effective. The compounds are administered one to three times daily at a level to achieve a total daily dose of about 0.4 to about 80 mg/kg/day. The precise amount of a compound used in the present method which is therapeutically effective, and the route by which such compound is best administered, is readily determined by one of ordinary skill in the art by comparing the blood level of the agent to the concentration required to have a therapeutic effect.

In this invention, in one embodiment, in one aspect a compound that can induce lipolysis can be used as an anti-obesity drug in the treatment of obesity to reduce the storage of fat in the body. In another aspect, a compound that can induce lipolysis can be used as an anti-obesity drug in the treatment of obesity to break down existing lipids in fat cells. In another aspect, a compound that can induce lipolysis can be used as an anti-obesity drug in the treatment of obesity to reduce the fat content of cells. The body mass index of a human or animal patient who is overweight can be reduced from the range between 30 kg/m² to 25 kg/m² to about 24 kg/m2 by a method of treatment comprising administration to the patient of a compound that can induce lipolysis in adipocytes of the patient. The body mass index of a human or animal patient who is obese can be reduced from the range above 30 kg/m² to a range between 30 kg/m² to 25 kg/m² and more preferably to about 24 kg/m2by a method of treatment comprising administration to the patient of a compound that can induce lipolysis in adipocytes of the patient.

In this invention, in another embodiment, in one aspect a compound that can block differentiation of a cell into an adipocyte, for example a compound that can block the differentiation of a preadipocyte cell into an adipocyte, can be used as an anti-obesity drug and in the treatment of overweight conditions and obesity to reduce the storage of fat in the body. In another aspect, a compound that can block differentiation of a cell into an adipocyte can be used as an anti-obesity drug and in the treatment of overweight conditions and obesity to prevent cells from accumulating fats or lipids in adipocytes or fat cells. In another aspect, a compound that can block differentiation of a cell into an adipocyte can be used as an anti-obesity drug in the treatment of overweight conditions and in the treatment of obesity to reduce the fat content of cells. The body mass index of a human or animal patient who is overweight can be reduced from the range between 30 kg/m² to 25 kg/m² to about 24 kg/m2 by a method of treatment comprising administration to the patient of a compound that can block differentiation of a cell into an adipocyte of the patient. The body mass index of a human or animal patient who is obese can be reduced from the range above 30 kg/m² to a range between 30 kg/m² to 25 kg/m² and more preferably to about 24 kg/m2 by a method of treatment comprising administration to the patient of a compound that can block differentiation of a cell into an adipocyte of the patient.

Pharmaceutically acceptable vehicle” means a carrier, diluent, adjuvant, or excipient, or a combination thereof, with which a component is administered to a patient. A pharmaceutically acceptable vehicle may include, but is not limited to, polyethylene glycol; wax; lactose; glucose; sucrose; magnesium stearate; silicic derivatives; calcium sulfate; dicalcium phosphate; starch; cellulose derivatives; gelatin; natural and synthetic gums such as, but not limited to, sodium alginate, polyethylene glycol and wax; suitable oil; saline; sugar solution such as, but not limited to, aqueous dextrose or aqueous glucose; DMSO; glycols such as, but not limited to, polyethylene or polypropylene glycol; lubricants such as, but not limited to, sodium oleate, sodium acetate, sodium stearate, sodium chloride, sodium benzoate, talc, and magnesium stearate; disintegrating agents, including calcium carbonate, sodium bicarbonate, agar, starch, and xanthan gum; and absorptive carriers such as, but not limited to, bentonite and klonin.

“Therapeutically effective amount” means the amount of a component that is sufficient to at least partially effect a treatment of a condition when administered to a patient. The therapeutically effective amount will vary depending on the condition, the route of administration of the component, and the age, weight, etc. of the patient being treated.

By systemic administration is meant oral, while it is possible for an active ingredient to be administered alone as the raw chemical, it is preferable to present it as a pharmaceutical formulation. The active ingredient may comprise, for oral administration, from 0.01 to 5.0 wt % of the formulation.

For oral administration of one active ingredients in 5% dextrose in water or normal saline, or a similar formulation with suitable excipients, is most effective. Typically, the parenteral dose will be about 0.01 to about 50 mg/kg; preferably between 0.1 and 20 mg/kg, in a manner to maintain the concentration of drug in the plasma at a concentration effective. The compounds are administered one to three times daily at a level to achieve a total daily dose of about 0.4 to about 80 mg/kg/day. The precise amount of a compound used in the present method which is therapeutically effective, and the route by which such compound is best administered, is readily determined by one of ordinary skill in the art by comparing the blood level of the agent to the concentration required to have a therapeutic effect.

Compounds that block differentiation of cells to adipocytes (fat cells) would be useful in that they would prevent cells from accumulating fat/lipids. In effect, fat would not be stored when cells have all the energy they require.

A fruit from the genus Garcinia shows lipolytic activity in adipose tissue. In rat livers, garcinia extract can inhibit fatty acid synthesis and subsequent lipid accumulation. Garcinia extract is currently used as a dietary supplement in the health food industry.

Leptin, a product of the obese gene, is a hormone secreted by mature adipocytes that may play a role in regulating body fat stores, energy expenditure and food intake. Leptin expression is regulated by physiological states such as fasting and feeding and by hormonal regulation as well as cytokines like TGF-alpha.

Insulin-stimulated glucose metabolism also plays a role in regulating leptin secretion. Glucose metabolism induced by insulin, rather than insulin itself per se, can increase the transcriptional activity of a promotor for leptin in human adipocytes as well as human subjects. Compositions that induce similar or higher levels of leptin secretion compared to insulin-induced leptin secretion) could be potentially useful drugs clinically.

Obesity and diabetes are inter-related and operate through similar/overlapping mechanisms and pathways. Because of the complexity of these metabolic disorders (and the multiple cell signaling pathways involved in each), discovery of new compounds to treat these disorders can be difficult. In order for a unique compound/extract to be taken to animal studies, which are time consuming and expensive, and then through drug development stages, solid mechanism based evidence must be gathered. Since there are multiple mechanisms/pathways involved in the onset of diabetes and obesity, a single assay/approach may be limiting for discovery of new chemical entities. Additionally the mechanism can be unknown. Therefore, we have taken a novel approach to this problem. I have developed a battery of assays to be used to quantify the ability of a test compound or extract of a plant to effect a mechanism related to the production or maintenance of obesity and potentially also of diabetes.

I have discovered that novel dietary supplements can be used for the treatment of disorders of fat tissue such as obesity and diabetes. Obesity can be treated effectively and safely at the level of the fat cell, rather than indirectly by modulating central nervous system (CNS). With this approach, selective agents can be identified for use in the treatment of disorders of fat tissue such as obesity and diabetes, which agents do not exhibit CNS or cardiovascular side effects. I have developed unique, cellular, HTP-screening assays using the culture of mouse adipocytes and preadipocytes to identify agents that are useful in the treatment of obesity. These assays allow one skilled in the art to focus on the detection of potential botanicals which target human fat tissue. These assays are primary cellular screens and follow-up response screens and engineered cellular screens. These assays can be used at an early stage in a drug discovery process with a strategy to maximize screening capacity to identify relevant products that are useful in the treatment of overweight conditions and obesity, and which are useful to lower the BMI index of an overweight or obese person toward and to the level of a healthy individual with a BMI index of 24 kg/m².

An array of cell-based assays has been developed and validated to detect and discover novel compounds and compositions (referred to as weight loss compounds) that exhibit activity in the treatment of overweight and obese conditions and which, when administered to a patient in need of treatment, result in weight loss in an overweight or obese person. It is an advantage of this invention that these weight loss compounds and compositions can be easily and readily identified by their relative activities against reference assay results and against results of assays employing a reference compound of known activity employing these assays in order to rapidly screen fractions of plants for new sources of safe and effective herbal ingredients. It is another advantage of this invention that active compounds are discovered in the extracts of plants. In addition, it is an advantage of this invention that rather than randomly searching a compound library, focused screening assays toward plant extracts to discover individual compounds and mixtures that are derived from plants which have been associated with ethnobotanical information and a history of safe usage. It is another advantage of this invention that assays have been identified to permit the evaluation of candidate plants with maximum efficiency, greatly reducing research and development time and expense.

Extracts from ethnobotanical plants can be conveniently chemically modified, for example, such as by treatment with acylating reagents such as organic acid halides or anhydrides or active esters to provide acylated extracts that can be screened in the assays to identify additional compounds that are useful in this invention. Additional methods of chemical modification of extracts include hydrogenation of some or all of olefinic groups in extracts, oxidation of alcohols, esterification by peroxidation, epoxide formation, ozonolysis, nitration, sulfination, sulfonation, sulfate formation, and phosphorylation. The adipocyte differentiation blocking assay is an enzyme assay that examines the ability of compounds to block differentiation of preadipocytes to adipocytes by quantifying the level of GPDH in cell lysates (inhibition of adipogenesis). Based on preliminary results using this assay, several plant extracts (designated as PM901, and as PM908, etc.respectively) and collected eluant fractions isolated by HPLC, the extracts being divided into 40 or 120 fractions in collection wells (collected at 30 second or 1 minute HPLC eluant flow time intervals). The cellular differentiation assay results indicate adiopogenesis blocking activity to be present in those fractions (or wells) that contained cells that did not differentiate into adipocytes after being exposed to a standard MDI treatment. MDI treatment is defined as Differentiation media causes the 3T3-L1 preadipocytes (fibroblast cells) to differentiate into adipocytes (fat cells). Differentiation media is composed of:
Per 100 ml of differentiation media
90 mL DMEM media
10 mL FCS
1% pen/strep
1 ml methylisobutylxanthine (stock 11.5 mg/mL)
100 ul dexamethasone (stock 0.4 mg/mL)
1 ml insulin (stock 1 mg/mL)

The marker compound in these samples can be identified for additional assay guided fractionation.

In addition, we also screen for substances that will cause adipocytes to revert their phenotype back to preadipocytes, that is, we also screen for substances that will cause adipocyte de-differentiation, by assessing the GPDH enzyme levels in treated cells. Multiple treatments with TNF-alpha cause dedifferentiation of adipocytes. A commercially available ELISA kit can be used to detect compounds that induce the release of leptin. And finally, to assess the effects of our plant extracts on lipolysis, an assay dubbed GPO Trinder assay available from Sigma-AldrichChemical Company is used. This assay detects glycerol released into a cell medium as a result of triglyceride (fat) breakdown.

We have found that active compounds of the present invention include cucurbitacin B and cucurbitacin D and cucurbitacin-like compounds. Cephalandrine A and B and Cephandrine-like compounds are triterpene compounds. Preferred compounds in this invention are found in the extracts obtained from ethnobiological plants.

The active compounds of this invention typically are cosmetically or pharmaceutically acceptable analogs, derivatives, or salts of cucurbitacins and Cephalandrine. In the practice of the present invention, the active compounds may alternatively be substituted with alkyl (both unsaturated and saturated, and branched and unbranched, such as methyl, ethyl, or isopropyl), aryl, halogen, hydroxy, alkoxy, and amino groups, as will be apparent to those skilled in the art. Additionally, any of the active compounds of the present invention may be present as an optical isomer, or chiral compound, or as a mixture of optical isomers and chiral compounds. These isomers may be isolated in pure form or enriched, for example, as a 50:50 racemic mixture of two isomers enriched to up to 100% of one isomeric pure form. Individual isomers or mixtures of isomers can be useful in this invention. The net activities of a mixture of one or more isomers will be observed in the assays of this invention.

Cucurbitacin is an important bioactive triterpene obtained from Coccinia grandis. This triterpene is not widely distributed and the most convenient sources are aerial parts of C. grandis and Cucurbita andreana.

Cucurbitacins B and D is obtained by solvent extraction of Cucurbiat andreana. U.S. Pat. No. 5,925,356 describes a process for the isolation of cucurbitacin B and D.
Analytical HPLC: For high-pressure liquid chromatography analysis an HP 1090 liquid chromatogram was used with a DR-5 solvent delivery system, variable volume autoinjector, autosampler, HP 1090 series II diode array detector system and MS-DOS software. Three detection wavelengths (220, 254, and 340 nm) were used to scan the samples. A 100×2.1 mm column filled with 5 um of C18 reverse phase gel was eluted with water and a linear gradient of 10-90% acetonitrile in 20 min at a flow rate of 0.5 ml/min. The HPLC profile of the crude extract, standardized extract and the marker compound have been recorded
Confirmation and identification of Marker compounds by Direct probe and Electrospray mass spectra: cucurbitacin B and D were first analyzed by direct probe/electron impact mass spectromety (EI MS). The results from these analysis could not prove conclusively the identification of these samples as B and D. A second type ionization was used to obtain the molecular ion. Two types of ‘soft’ ionization were used; Electrospray (ES) and atmosphere pressure ionization (API). Both of these techniques can give a protonated molecular ion, [M+H]+. API did not give the expected results but rather the lose of the acetyloxy and or OH groups giving a major ion at 499 with sequential lose of the OH groups I. e., ions at m/z 481, 463 and 445. Both cucurbitacin B and D gave similar ions and could not be distinguished from each other.

ES analysis does give a molecular ion for both cucurbitacins B and D. The cucurbitacin B gave ions at m/z 559.2 and 618.3 corresponding to [m+H]+ and [M+H2O+CH3CN]+, respectively. The cucurbitacin D gave ions at m/z 517.3 and 576.3 corresponding to [M+H]+ and [M+H2O+CH3CN]+, respectively. These molecular weights correspond to published literature values. Other three compounds isolated are, Cephalandrol, Cephalandrine A and B

Test compounds in this invention can be individual compounds or mixtures of individual compounds or extracts of plants or fractionated extracts of plants or purified extracts of plants, any of which can be herein referred to as a test compound.

An objective of the assays used in this invention is to discover and identify or find and isolate compounds or mixtures of compounds that can be used to treat obesity. Adipocytes play a role in the storage of energy in the form of lipids, and serve as endocrine cells that regulate the breakdown and synthesis of lipids. Fully developed adipocytes synthesize and accumulate triglycerides, and produce high levels of the enzyme glycerol-3-phosphate dehydrogenase (GPDH). In addition, the breakdown of triglycerides (lipolysis) leads to the secretion of fatty acids and glycerol in the cell environment.

Fully developed adipocytes synthesize and accumulate triglycerides. The breakdown of triglycerides (i.e., lipolysis) leads to the secretion of fatty acids and glycerol in the cell environment. Compounds identified in this invention that stimulate lipolysis, i.e. the break down existing stored triacylglycerol, can be used to reduce the storage of fat in the body, and subsequently to reduce body weight. Additionally, lipolytic compounds may break down existing lipids/fat cells, or at least reduce the fat content of cells.

Compounds identified in the assays of this invention that block or inhibit adipogenesis (i.e., the differentiation of preadipocytes to adipocytes) are useful in preventing the development of cells whose function is the accumulation and/or synthesis of stored triacylglycerol/fat. As the adipocyte secretes a number of inflammatory cytokines, blocking the differentiation process would also result in an overall lack of inflammatory mediators such as TNF-α, whose presence is correlated with insulin resistance. Therefore, inhibitors of adipogenesis will prevent the accumulation of fat as well as reduce the insulin resistance associated with diabetes. We assess inhibition of adipogenesis using the following assay methods.

Method i): Oil Red O staining which stains fat droplets in fat cells and thereby gives a directly related indication of the level of adipogenesis as a function of the amount of staining of fat droplets.

Method ii): GPDH or glyceraldehyde phosphate dehydrogenase is a key enzyme involved in the pathway of triglyceride synthesis in the glycolytic pathway. Adipocytes express highlevels of this enzyme. Therefore, a reduction in GPDH levels is indicative of inhibition of adipogenesis (prevention of fat accumulation in cells).

Method iii): Assay to observe de-differentiation of adipocytes to preadipocytes can be used to detect compounds and mixtures of compounds that are capable of breaking down existing stores of body fat. This assay identifies compounds and mixtures of compounds than induce the differentiation of adipocytes to preadipocytes and is quantitatively assessed by developing a color response using Oil Red O staining or by measuring GPDH levels as in methods i) and ii), respectively. Such compounds and mixtures of compounds as extracts of plants can have a direct effect on reducing weight because they cause fat cells to become non-fat cells (preadipocytes).

Method iv) Leptin Secretion assay can be used to determine the presence of leptin which is a product of the Ob gene. It is a hormone secreted by mature adipocytes that plays a role in regulating body fat storage. High levels of leptin circulating in the blood indicate hormonally that energy stores are high, and therefore mitigate the need for further food intake. Compounds or mixtures of compounds of this invention that increase leptin secretion can be administered orally to an overweight or obese person to reduce appetite in that person, and therefore to reduce food intake by that person.

The assays of this invention are described hereinbelow.

Assay 1. Lipolysis assay.

A calorimetric assay is used to screen for compounds and mixtures of compounds that induce lipolysis or the breakdown of lipids.

In this assay, pre-adipocyte cells are seeded in 96-well plates and are induced to convert to adipocytes over the course of a 10-15 day treatment with lipolytic reagents. After cells have converted to adipocytes, the cells are starved, and then subsequently treated with or exposed to test compounds or mixtures of compounds or extracts of plants or fractionated extracts of plants. The adipocytes are treated with test compounds in the presence of a surrounding cell medium. Over the course of 1 to 3 hours after exposure to the test compounds or mixture of compounds, the amount of glycerol present in the cell medium is measured. Glycerol and fatty acids are hydrolysis products of triglycerides, and glycerol is released from cells after enzymatic hydrolysis or breakdown of lipids. Results are expressed as a percentage of the amount of glycerol present detected in a reference assay comprising an untreated control. Untreated controls are cells that are treated with vehicle only (such as 100% DMSO). A compound that is active in the mechanism of lipolysis of a triglyceride can exhibit at least 120% to 150% of the amount of glycerol released relative to an untreated control.

Assay 2. Inhibition of adipocyte differentiation.

Inhibition of adipocyte differentiation is assayed by measuring the levels of the enzyme glycerol-3-phosphate dehydrogenase (GPDH). GPDH is involved in the pathway of triglyceride synthesis in the glycolytic pathway. Colorimetric assays can be used to screen for compounds and mixtures of compounds than inhibit the differentiation of preadipocytes to adipocytes. Oil Red O is a colorimetric assay that can be used to stain lipids in adipocytes.

In this assay pre-adipocyte cells require 10 to 15 days to differentiate into adipocytes. Pre-adipocyte cells are seeded in 96-well plates and induced to convert to adipocytes over 10-15 days. On day 1 and every 3 days thereafter test compounds are added to the cells to determine the extent to which the compounds block the conversion of preadipocytes to adipocytes. After cells have undergone the 10-15 days required to differentiate, cells are assessed for GPDH activity.

Results are expressed as a percentage of the untreated control average. Untreated controls are cells that are treated with vehicle only (100% DMSO), and have high levels of GPDH activity (typical of adipocytes). Compounds that do block differentiation will cause cells to show little or no GPDH activity. A lead compound in the GPDH assay would exhibit a 40-50% or more decrease in GPDH expression when compared to the untreated controls.

For the Oil Red O assay, cells that do not take up the red dye have failed to convert to adipocytes. Compounds or mixtures of compounds evaluated in this assay are positive or good leads when cells do not take up red dye. Compounds that block differentiation of cells to adipocytes (fat cells) would be useful in that they would prevent cells from accumulating fat/lipids. In effect, fat would not be stored when cells have all the energy they require. These compounds can be used to treat obesity and over weight conditions.

Assay 3. De-Differentiation of adipocytes to preadipocytes. This assay comprises a colorimetric assay to screen for compounds and mixtures of compounds that induce the differentiation of adipocytes to preadipocytes. The presence of such compounds is determined by measuring the relative levels of the enzyme GPDH. In this assay, pre-adipocyte cells are seeded in 96-well plates and induced to convert to adipocytes over the course of a 10-15 day treatment with lipolytic reagents. After cells have fully differentiated to adipocytes they are treated with compounds or mixtures of compounds to be screened over the next 10-15 days to convert the cells ‘back’ to preadipocytes and measure the efficacy of the compounds to de-differentiate the adipocytes back to preadipocytes. GPDH levels present in the cells are measured to determine if the cells still express the adipocyte phenotype. Results are expressed as GPDH levels present in the cells as a percentage of GPDH levels present in an untreated control average, the untreated control comprising adipocytes, i.e., adipocyte cells that are treated with vehicle only (100% DMSO). A compound useful in the treatment of conditions of overweight and obesity and related disorders would show GPDH levels 30-50% lower than untreated control in this assay, and would be useful as an anti-obesity reagent. Compounds or mixtures of compounds that induce lipolysis can be useful when administered to a body, preferably by an oral route, to reduce the storage of fat in the body. Additionally, such compounds and mixtures of compounds can be useful to break down existing lipids in fat cells and reduce the fat content of cells. Treatment of an overweight person or obese person can reduce that person's body mass index from levels identified as above normal to lower levels, preferably to levels in a normal range.

Assays useful to discover compounds and mixtures of compounds in this invention are described in the following.

### Protocol For 96-well Adipogenic Differentiation Assay (GPDH Assay)

L1 cell differentiation is obtained in a process comprising the following steps.
1) 5,000 to 10,000 cells per well of L1 cells are seeded in poly-D-lysine-treated plates containing an array of wells (e.g., 96 wells per plate) which are incubated at 30 to 37° C. in an atmosphere comprising carbon dioxide, oxygen, and nitrogen.
2) If the cells are 100% confluent 24 hours after seeding, the plates are incubated at the same conditions for an additional 48 hours;

2 days after confluent (three days after seeding) the cells are treated with MDI (i.e., MDI is Differentiation media causes the 3T3-L1 preadipocytes (fibroblast cells) to differentiate into adipocytes (fat cells). Differentiation media is composed of:
Per 100 ml of differentiation media
90 mL DMEM media
10 mL FCS
1% pen/strep
1 ml methylisobutylxanthine (stock 11.5 mg/mL)
100 ul dexamethasone (stock 0.4 mg/mL)
1 ml insulin (stock 1 mg/mL)
   3 and add TNF-alpha (5, 10 ng/ml) to wells used as control wells with MDI;
   4) 2 days later the media is removed and replaced with fresh media + units of insulin;
   5) 3 days later the media is removed and replaced with media +¼ of the amount of insulin used in the previous step;
   6) 2 days later the media is removed and replaced with media only; wherein
   7) the cells are fully differentiated 3 days later (after total of 11 days post MDI treatment).
   The following are used as controls.
   A negative control comprises addition of 0.1 mls of TNF-alpha at a concentration of 5 ng/ml which is added to at least one of the wells as a control on the same day as MDI treatment.
   Positive Control: MDI treatment

### GPDH Assay

Cells to be assayed are prepared for GPDH assay in a process comprising the following steps.
   1) Wash cells in each well once (1X) with PBS (phosphate buffered saline that is free of calcium and magnesium);
   2) Add ice-cold homogenization solution to each well using 100 ul of solution per well
   3) Store plates at −20° C. to break up the cells and release GPDH.

The GPDH assay comprises an enzyme reaction in a process comprising the following steps.
add 90 uL (microliters) of enzyme reaction mix comprising Enzyme Reaction Mix composition:
0.1 M triethanolamine
2.5 mM EDTA
0.1 mM Beta-mercaptoethanol
334 uM NADH
pH 7.7 (using HCl)
   1) to each well and pre-incubate for 10 minutes at 37° C.;
   2) add 10 ul (microliters) of DHAP which is defined as Dihydroxyacetone phosphate (at a concentration of 4 mM of stock solution in H₂O) to start the assay;
   3) measure absorbance in each well at 340 nm for 4-5 minutes
   Controls comprise the following separate control wells in which absorbance at 340 nm is also measured for 4-5 minutes:
      1 well w/o (without) NADH, but which receives PBS buffer only
      at least one well used with No MDI treatment
      at least one well used with TNF-alpha treatment
      at least one well used with MDI treatment only with vehicle addition;
      at least one well used with MDI treatment only without vehicle addition.
A homogenization solution is prepared comprising the following ingredients and pH:
20 mM Tris
1 mM EDTA
1 mM Beta-mercaptoethanol
pH 7.3
An enzyme reaction mixture is prepared comprising the following ingredients and pH:
0.1 M triethanolamine
2.5 mM EDTA
0.1 mM Beta-mercaptoethanol (optionally using DTT or 1,4-Dithio-DL-threitol instead of beta-mercaptoethanol)
334 uM NADH
pH 7.7 (using HCl)
A useful protocol for the de-differentiation assay in this invention comprises the following steps.
L1 cell differentiation
   1) Seed 5-10-K/well of L1 cells in poly D lysine treated plates
   2) 100% confluent next day. Continue to incubate for 2 more days
   3) 2 days after confluent treat with MDI
      add TNFalpha with MDI
      add compounds to be tested at this time as well
   4) 2 days later remove media and add media + insulin
   5) 3 days later remove media and add media +¼ insulin
   6) 2 days later and media only
   7) cells fully differentiated 3 days later (after total of 11 days post MDI treatment)
   controls:
   Negative: TNFalpha (5 ng/ml)
      added to wells on the same day as MDI treatment
Positive Control: MDI treatment
Induce Cells to revert back to preadipocytes
   1) administer TNF-alpha to induce reversion back to preadipocytes
GPDH assay
   1) carry out same as previously, but this time measuring a “lack” of GPDH activity as anindication of blockage of lipogenesis.

The invention is further described by reference to the figures.

FIG. 6 shows the effect of AdipoCleave on lipolysis in 12-day old differentiated adipocytes. Cells were treated with 5 or 50 ug/ml of cucurbitacin B or D for 3 hours. Conditioned media was then collected and assessed for glycerol release using GPO trinder assay (Sigma). Results are expressed as a percentage of the untreated control average. Untreated controls are cells that are treated with vehicle only. Results are presented as the mean ± SD of three replicated experiments.

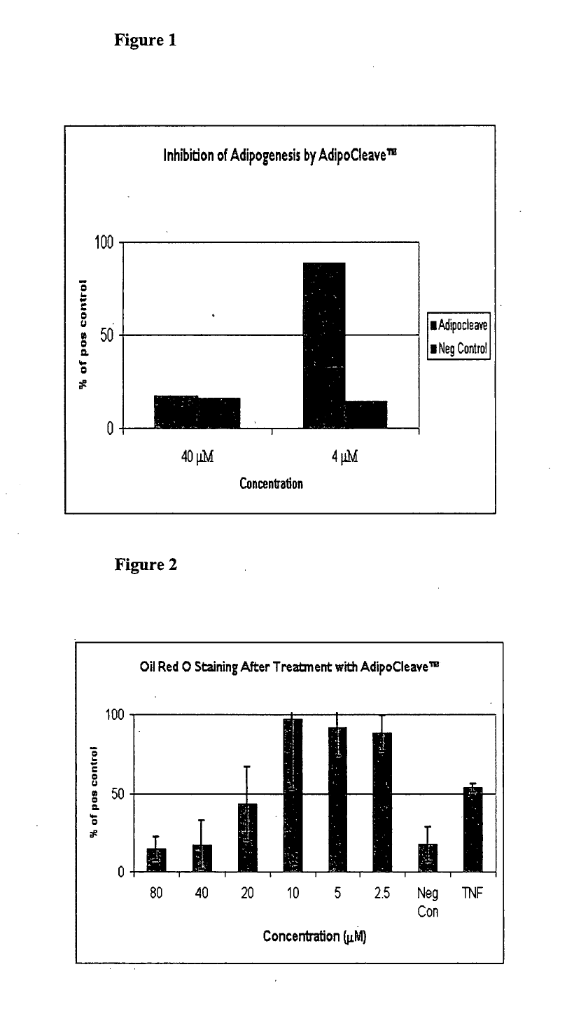

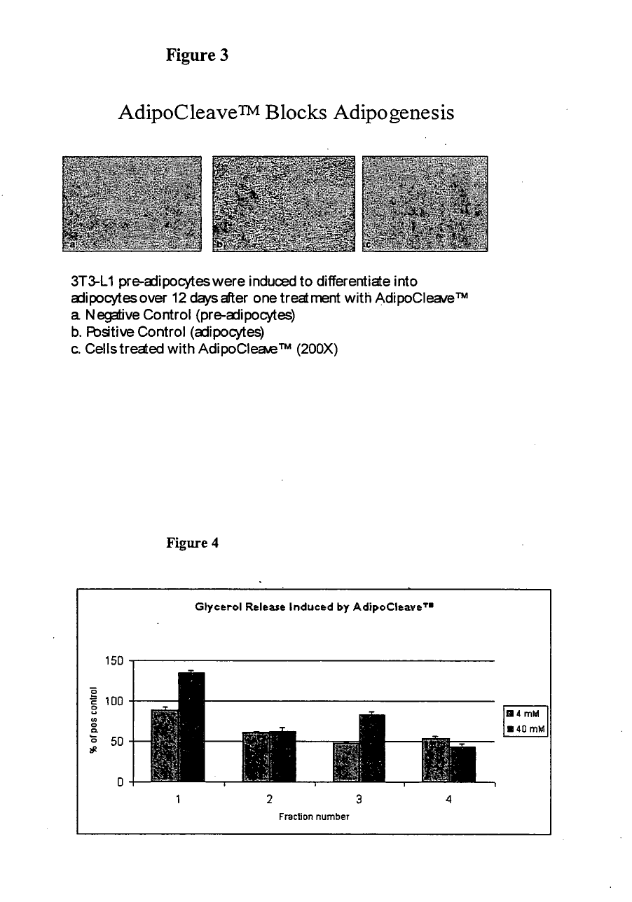

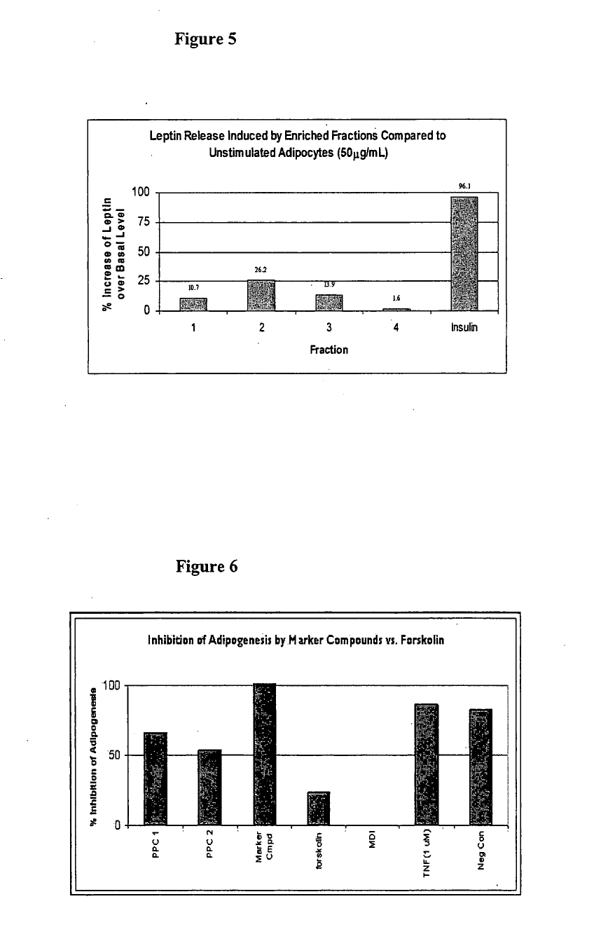

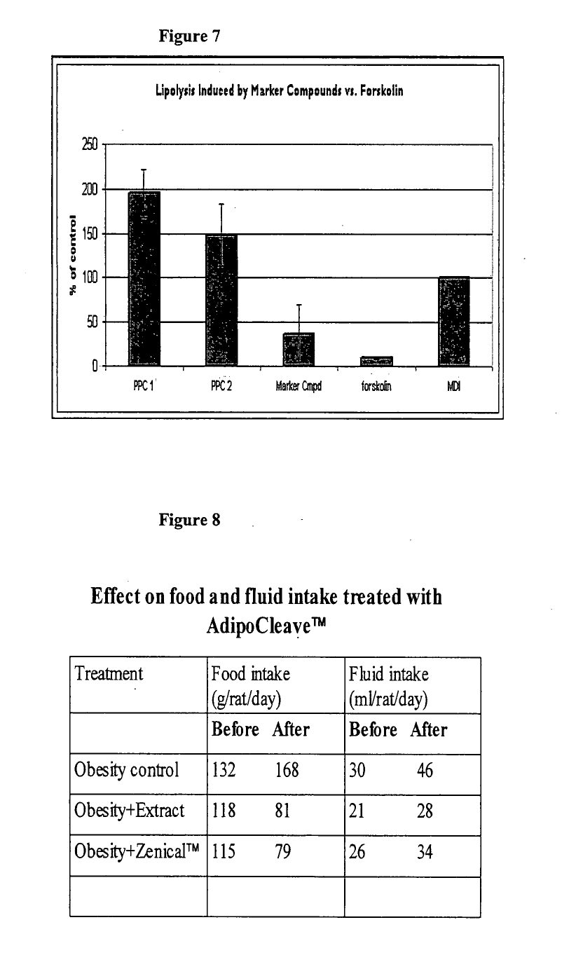

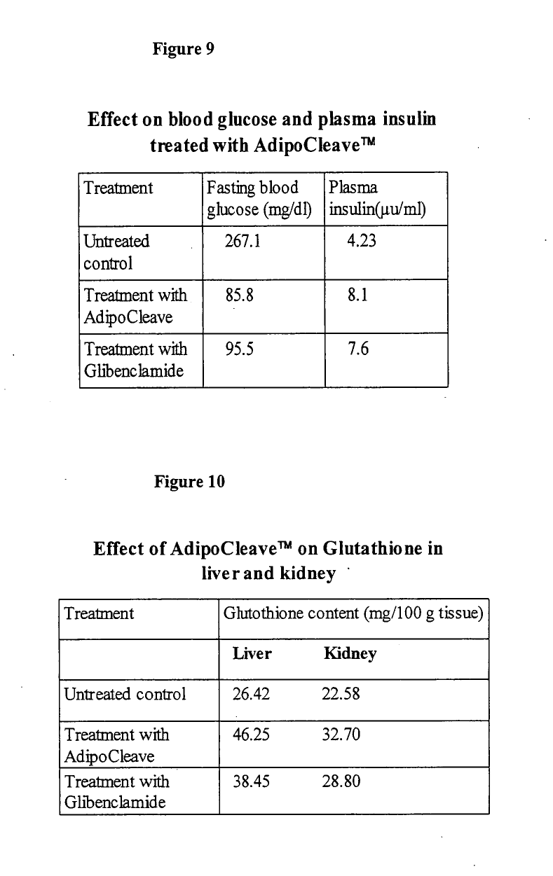

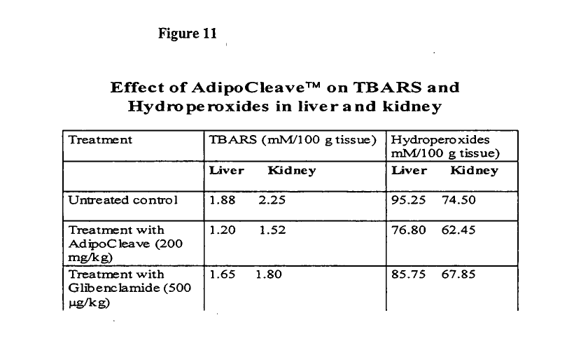

FIG. 1 shows the effect of AdipoCleave on adipogenesis. 3T3-L1 cells were treated with 5 or 50 ug/ml of cucurbitacin B or D three times during the course of differentiation into adipocytes. After 12 days, GPDH levels were measured. Results are expressed as percent inhibition compared to the positive control average (positive control is shown here as 0% inhibition). Results are presented as the mean ± SD of three replicated experiments.

Compositions comprising compounds or mixtures of compounds or extracts of plants can be assayed by the above methods and identified to be useful in the treatment of over weight conditions and obesity and related disorders.

Compositions that are active in the assays of this invention can be formulated with pharmaceutically acceptable or nutraceutically acceptable excipients and ingredients to form oral dosage forms. Suitable oral dosage forms include liquid solutions such as a solution comprising a vegetable- and/or animal- and/or fish-derived oil, optionally also containing at least one pharmaceutically acceptable surface active agent, optionally also containing a low molecular weight hydroxy compound such as ethanol and/or glycerol, and the solution enclosed in a soft gelatin capsule for oral administration. The active component can be present in amounts from about 0.1% to about 50% of the solution, preferably from about 2% to about 40% of the solution, and more preferably from about 5% to about 30% of the solution. Solid compounds of this invention can also be formulated into tablets and capsule for oral administration or used as powders for addition to foodstuff prior to consumption by a patient. Coloring and flavoring ingredients acceptable for food or pharmaceutical use can be used in such formulations.

Compositions that are active in the assays of this invention include cucurbitacin B, D and Cephalandrol, Cephalandrine A and B as well as cucurbitacin-like or cephalandrol-like compounds and plant extracts containing these compounds.

A method of treatment of obesity in a patient with a body mass index of 30 kg/m² or greater comprises administration to the patient of cucurbitacins or Cephalandrol, Cephalandrine A and B.

A method of treatment of an overweight condition in a patient comprises administration to the patient of cucurbitacins or Cephalandrol, Cephalandrine A and B, wherein the body mass index of the patient is reduced from the range of about 30 kg/m² to about 25 kg/m², to about 24 kg/m².

Pharmaceutically acceptable vehicle” means a carrier, diluent, adjuvant, or excipient, or a combination thereof, with which a component is administered to a patient. A pharmaceutically acceptable vehicle may include, but is not limited to, polyethylene glycol; wax; lactose; glucose; sucrose; magnesium stearate; silicic derivatives; calcium sulfate; dicalcium phosphate; starch; cellulose derivatives; gelatin; natural and synthetic gums such as, but not limited to, sodium alginate, polyethylene glycol and wax; suitable oil; saline; sugar solution such as, but not limited to, aqueous dextrose or aqueous glucose; DMSO; glycols such as, but not limited to, polyethylene or polypropylene glycol; lubricants such as, but not limited to, sodium oleate, sodium acetate, sodium stearate, sodium chloride, sodium benzoate, talc, and magnesium stearate; disintegrating agents, including calcium carbonate, sodium bicarbonate, agar, starch, and xanthan gum; and absorptive carriers such as, but not limited to, bentonite and klonin.

“Therapeutically effective amount” means the amount of a component that is sufficient to at least partially effect a treatment of a condition when administered to a patient. The therapeutically effective amount will vary depending on the condition, the route of administration of the component, and the age, weight, etc. of the patient being treated.

By systemic administration is meant oral, while it is possible for an active ingredient to be administered alone as the raw chemical, it is preferable to present it as a pharmaceutical formulation. The active ingredient may comprise, for oral administration, from 0.01 to 5.0 wt % of the formulation.

For oral administration of one active ingredients in 5% dextrose in water or normal saline, or a similar formulation with suitable excipients, is most effective. Typically, the parenteral dose will be about 0.01 to about 50 mg/kg; preferably between 0.1 and 20 mg/kg, in a manner to maintain the concentration of drug in the plasma at a concentration effective. The compounds are administered one to three times daily at a level to achieve a total daily dose of about 0.4 to about 80 mg/kg/day. The precise amount of a compound used in the present method which is therapeutically effective, and the route by which such compound is best administered, is readily determined by one of ordinary skill in the art by comparing the blood level of the agent to the concentration required to have a therapeutic effect.

A method of treatment of obesity in a patient comprises administration to the patient of cucurbitacins or Cephalandrol, Cephalandrine A and B, wherein the body mass index of the patient is reduced from a value greater than 30 kg/m² to a value of 30 kg/m² or less.

A process for the identification of a composition or compound useful in the treatment of an overweight or obese person, comprises an assay comprising:

a) obtaining an extract of an ethnobotanical plant, and

evaluating the activity of the extract in an assay selected from the group consisting of a lipolysis assay, an assay that measures the amount of glycerol introduced by a cell into a suspension medium of the cell, an adipocyte differentiation assay, an assay that measures the level of the enzyme glycerol-3-phosphate dehydrogenase, an assay that measures the inhibition of differentiation of preadipocytes to adipocytes, an assay that measures the accumulation of lipid in an adipocyte, an assay that measures the de-differentiation of adipocytes into preadipocytes, and combinations thereof.

A schematic representation of a commercially available ELISA assay that can be used to detect compounds that induce the release of leptin in this invention
Leptin procedure in flowchart form
Tissue Culture
1. Seed 2.5×10⁴ 3T3-L1 cells per well in a 48-well plate
2. After cell become confluent (2-3 days), differentiate 3T3-L1 pre-adipocytes into mature adipocytes over the course of 12-15 days
3. Rest cells-Replace cell media with 150 ul of Basal media (serum free DMEM with 1% BSA) for 3 days.
4. Add samples and controls (1 or 10 ug/well) to wells. Positive control is
5. Collect cell supernatant after 3 days, centrifuge and take 50 ul of supernatant for analysis using Leptin ELISA kit using protocol suggested by manufacturer (R&D Systems cat#MOB00).

Having thus described the invention, it will become better understood from the applied claims where it is set forth in a non-limiting manner.

What I claim as my invention is:

1. A method of treatment of diabetes and obesity in a patient with a body mass index of 30 kg/m² or greater, comprising administering to the patient an effective amount of C. grandis standardized extract (AdipoCleave) with cucurbitacins B and D and Cephalandrol, Cephalandrine A and B as active ingredients, in an amount wherein the body mass index of the patient is reduced.

2. The method of treatment of claim 1, wherein the patient has a high blood sugar content similar to the conditions of diabetes, and overweight conditions in a patient, and further comprising administering to the patient of C. grandis standardized extract (AdipoCleave) with cucurbitacins B and D and Cephalandrol, Cephalandrine A and B as active ingredients, in an amount wherein the body mass index of the patient is reduced from the range of about 30 kg/m² to about 25 kg/m², to about 24 kg/m².

3. The method of treatment of claim 1, further comprising administering to the patient C. grandis standardized extract (AdipoCleave) with cucurbitacins B and D and Cephalandrol, Cephalandrine A and B as active ingredients, in an amount wherein the body mass index of the patient is reduced from a value greater than 30 kg/m² to a value of 30 kg/m² or less.

4. A healthy blood sugar maintenance composition comprising an effective amount of: C. grandis standardized extract (AdipoCleave) with cucurbitacins B and D as active ingredients, cucurbitacins and Cephalandrol, Cephalandrine A and B and related analogs

5. The healthy blood sugar maintenance composition of claim 4, further comprising an effective amount of approximately: 200 mg C. grandis standardized extract (AdipoCleave) with cucurbitacins B and D as active ingredients

6. A process for the identification of a composition or compound useful in the treatment of an diabetic and or overweight or obese person, comprising an assay comprising:
a) obtaining an extract of an ethnobotanical plant, and
b) evaluating the activity of the extract in an assay selected from the group consisting of a lipolysis assay, an assay that measures the amount of glycerol introduced by a cell into a suspension medium of the cell, an adipocyte differentiation assay, an assay that measures the level of the enzyme glycerol-3-phosphate dehydrogenase, an assay that measures the inhibition of differentiation of preadipocytes to adipocytes, an assay that measures the accumulation of lipid in an adipocyte, an assay that measures the de-differentiation of adipocytes into preadipocytes, and combinations thereof.
